(19) United States
(12) Patent Application Publication
(10) Pub. No.: US 2022/0096253 A1
(43) Pub. Date: Mar. 31, 2022

(54) ADJUSTABLE LUMEN APPOSING STENT

(71) Applicant: Boston Scientific Scimed, Inc., Maple Grove, MN (US)

(72) Inventors: Daniel Tuck, Galway (IE); Martyn G. Folan, Loughrea/Co. Galway (IE); Michael Rodgers, Mayo (IE); Martin Burke, Galway (IE)

(21) Appl. No.: 17/479,447

(22) Filed: Sep. 20, 2021

Related U.S. Application Data
(60) Provisional application No. 63/083,409, filed on Sep. 25, 2020.

(51) Int. Cl. A61F 2/88; A61F 2/848

(52) U.S. Cl. CPC A61F 2/885; A61F 2/848; A61F 2230/0095; A61F 2230/0091; A61F 2002/8486

(57) ABSTRACT

The present disclosure relates generally to the field of medical devices. In particular, the present disclosure relates to medical devices for facilitating the flow of fluids and materials in and/or between adjacent body lumens, for example, a stent which maintains an open flow passage between body lumens. In one example, a stent may have a constrained configuration and an expanded configuration. In the expanded configuration, the stent may have first and second retention members and saddle region extending therebetween. The saddle region may comprise a coil configured to apply a restoring force to tissue surfaces respectively apposed to the first and second retention members.

### PRIORITY

The present application is a non-provisional of, and claims the benefit of priority under 35 U.S.C. § 119 to, U.S. Provisional Application Ser. No. 63/083,409, filed Sep. 25, 2020, the disclosures of which are herein incorporated herein by reference in their entirety for all purposes.

### FIELD

The present disclosure relates generally to the field of medical devices. In particular, the present disclosure relates to medical devices for facilitating the flow of fluids and materials, such as a stent to maintain an open flow passage between or within body lumens.

### BACKGROUND

Placement of a self-expanding metal stent (SEMS) within an anatomical area (e.g., body lumen, passage, vessel, duct, etc.) may enable fluid communication from one area to another. For example, a stent may enable flow of fluid from one body lumen to another.

Stents are typically provided in incremental sizes, and a particularly sized stent is selected for use in a procedure based on the anatomical dimensions of the patient's tissue. An improperly sized stent may cause undue irritation to apposed tissues, for example, if a stent is too short to accommodate the width of tissues being held between retention members of the stent. In some cases, an improperly sized stent may prevent the formation of a seal between the stent and apposed tissues. As a result, a stent that is not optimally sized for the target patient anatomy may result in tissue damage and/or other unintended consequences, such as leakage of fluid around the stent, thereby presenting discomfort and/or dangerous risk to a patient.

With the above considerations in mind, a variety of advantageous medical outcomes may be realized by the devices and/or methods of the present disclosure.

### SUMMARY

In one aspect, a stent may comprise an elongate body having a constrained configuration. The elongate body may have an expanded configuration with a first portion expanded into a first retention member. The elongate body may have a second portion expanded into a second retention member. The elongate body may have a saddle region. The saddle region may define a lumen extending between the first and second retention members.

In the described and other aspects, the first retention member may comprise a first flange and a second flange configured to appose first and second surfaces of the first tissue, the second retention member may comprise a third flange and a fourth flange configured to appose first and second surfaces of the second tissue, or both. The first retention member may comprise a mesh structure, the second retention member may comprise a mesh structure, or both. In many embodiments, the coil may extend along an entire length of the saddle region. The coil may be configured to apply tension between the first retention member and the second retention member. The coil may be configured to apply a laterally outward force between the first retention member and the second retention member. The saddle region may comprise two or more coils. The two or more coils may be joined end-to-end. The two or more coils may be joined at a center point. Each of the two or more coils may comprise different spring constants. The saddle region may comprise at least one curve. The first retention member, the second retention member, and the coil may be continuously formed of wire. The first retention member may comprise a first surface substantially perpendicular to a longitudinal axis of the saddle region, the first surface configured to appose a first tissue, the second retention member may comprise a second surface substantially perpendicular to a longitudinal axis of the saddle region, the second surface configured to appose a second tissue, or both. The coil may comprise a shape memory material. The stent may comprise a cover.

In another aspect, a hollow body may comprise a constrained configuration and an unconstrained configuration. The hollow body in the unconstrained configuration may comprise a first retention member, a second retention member, and a central region therebetween. The central region may comprise a coil configured to, when deflected, generate a force to return towards a predetermined position. The first retention member and the second retention member may be configured to translate the force to tissue engaged by the first retention member, tissue engaged by the second retention member, or both.

In the described and other aspects, the coil may be configured to apply tension between the first retention member and the second retention member. The coil may be configured to apply a laterally outward force between the first retention member and the second retention member. The first retention member, the second retention member, and the coil may be continuously formed of wire.

In yet another aspect, a stent may comprise a first retention member configured to appose a first tissue layer, a second retention member configured to appose a second tissue layer, and a saddle region extending therebetween. The saddle region may comprise coil configured to affect a spacing between the first tissue layer and the second tissue layer.

In the described and other aspects, the coil may be configured to apply tension between the first retention member and the second retention member. The coil may be configured to apply a laterally outward force between the first retention member and the second retention member.

The first retention member, the second retention member, and the coil may be continuously formed of wire. The coil extends along an entire length of the saddle region.

### BRIEF DESCRIPTION OF THE DRAWINGS

Non-limiting examples of the present disclosure are described with reference to the accompanying figures, which are schematic and not intended to be drawn to scale. In the figures, each identical or nearly identical component illustrated is typically represented by a single numeral. For purposes of clarity, not every component is labeled in every figure, nor is every component in each embodiment of the disclosure shown where illustration is not necessary to allow those of skill in the art to understand the disclosure. In the figures:

FIGS. 1A-C illustrate side views of a self-expanding stent, according to one or more embodiments described herein.

FIGS. 2A-B illustrate a self-expanding stent according to one or more embodiments described herein.

FIG. 3 illustrates an exemplary use of a stent according to one or more embodiments described herein.

FIG. 4 illustrates an exemplary use of a self-expanding stent according to one or more embodiments described herein.

FIG. 5 illustrates an exemplary curved self-expanding stent according to one or more embodiments described herein.

FIG. 6 illustrates a further exemplary curved self-expanding stent according to one or more embodiments described herein.

FIG. 7 illustrates a side view of a self-expanding stent, according to one or more embodiments described herein.

### DETAILED DESCRIPTION

Placement of a self-expanding metal stent (SEMS) within an anatomical area (e.g., body lumen, passage, vessel, duct, etc.) may enable fluid communication from one area to another. For example, a stent may enable flow of fluid from one body lumen to another.

Stents are typically provided in incremental sizes, and a particularly sized stent is selected for use in a procedure based on the anatomical dimensions of the patient's tissue. For example, a particular stent may be selected to match the dimensions of one or more body lumens, the length of a desired passage intended to be formed by the stent, the distance between the tissues that will be apposed by the stent, or other dimensions.

As will be appreciated, it can be difficult for a physician to accurately ascertain the dimensions of the patient tissue so that an optimally sized stent can be selected. For example, tissue motion and/or lack of clear visibility of the tissue may detract from the physician's ability to select a properly sized stent for use in a procedure.

An improperly sized stent may cause undue irritation to adjacent tissues, for example, if a stent is too long and an end thereof extends beyond an apposed tissue through a body lumen such that it interacts undesirably with other tissue. In some cases, an improperly sized stent may prevent the formation of a seal between the stent and apposed tissues. For example, a stent may not have sufficient length to bridge tissue(s) effectively and may therefore tend to migrate, which can result in the failure to properly form a seal with the tissue(s) positioned between the stent's retention members. As a result, an improperly sized stent may result in tissue damage and/or other unintended consequences, such as leakage of fluid around the stent, thereby presenting discomfort and/or other symptoms to a patient.

With the above considerations in mind, a variety of advantageous medical outcomes may be realized by the devices and/or methods of the present disclosure. For example, a stent, according to one or more embodiments described herein, may have an automatically adjustable length in order to accommodate various applications. Accordingly, a physician may not need to approximate a desired stent size as is required with conventional stents.

The disclosure is not limited to the particular embodiments described, as such may vary. The terminology used herein is for the purpose of describing particular embodiments only and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to medical devices and systems and procedures associate with the gastrointestinal system, it should be appreciated that such medical devices and methods may be used with tissues of the abdominal cavity, digestive system, thoracic cavity, urinary and reproductive tract and the like.

As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

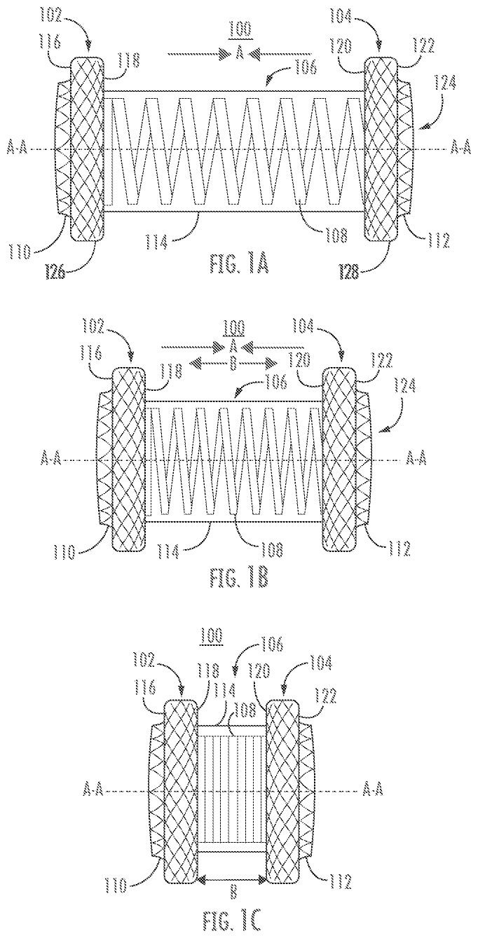

FIGS. 1A-C illustrate an exemplary stent according to one or more embodiments herein. Stent 100 may comprise first and second retention members 102, 104 coupled to each other via a saddle region 106 extending therebetween. FIG. 1A illustrates stent 100 in a first configuration (e.g., an expanded configuration), FIG. 1B illustrates stent 100 in an intermediate configuration (e.g., a partially expanded configuration), and FIG. 1C illustrates stent 100 in a second configuration (e.g., compressed configuration).

First and second retention members 102, 104 may each form a flange or other expanded portion having a diameter that is greater than a diameter of the saddle region 106. In many embodiments, one or both of first and second retention members 102, 104 may be configured to contact a tissue surface. For example, first retention member 102 may appose or otherwise engage a tissue surface (not shown) along a first axial outer surface 116, a first radial outer surface 126, and/or a first axial inner surface 118. Second retention member 104 may appose or otherwise engage a tissue surface (not shown) along a second axial outer surface 122, a second radial outer surface 128, and/or a second axial inner surface 120. Thus, in some embodiments stent 100 may be configured to press opposing tissue surfaces together, while in other embodiments stent may be configured to hold opposing tissue surfaces apart. For example, stent 100 may be deployed to appose two or more tissue walls along first and second axial outer surfaces 116, 122 or along first and second axial inner surfaces 118, 120. In another example, stent 100 may be deployed to engage tissue along first and second radial outer surfaces 126, 128 (e.g., within a single body lumen). First axial outer surface 116, first axial inner surface 118, second axial inner surface 120, and/or second axial outer surface 122 may each comprise a flat surface (e.g., a surface substantially perpendicular to a longitudinal axis A-A extending through the stent 100, or at an angle (not shown)) or they may comprise a surface or surfaces with one or more curves (not shown). First radial outer surface 126 and/or second radial outer surface 128 may comprise a flat surface (e.g., a surface substantially parallel to longitudinal axis A-A, or at an angle (not shown)) or a surface with one or more curves (not shown). First and second retention members 102, 104 may be formed of wire, one or more braids, a mesh structure, or the like, and may be formed of one or more metals, polymers, or other appropriate material. In many embodiments, the one or more of first and second retention members 102, 104 may comprise a shape memory material, such as Nitinol.

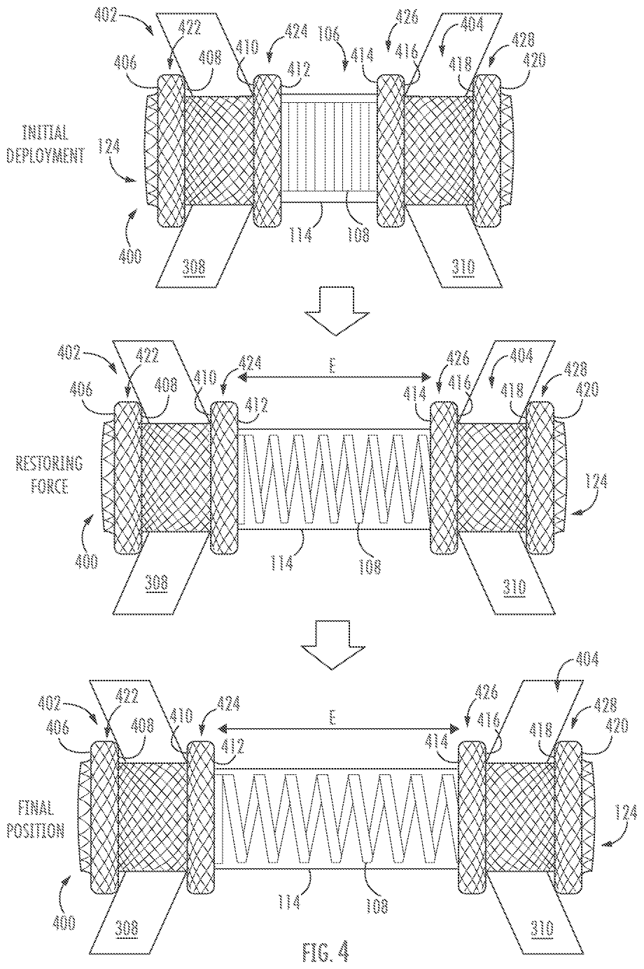

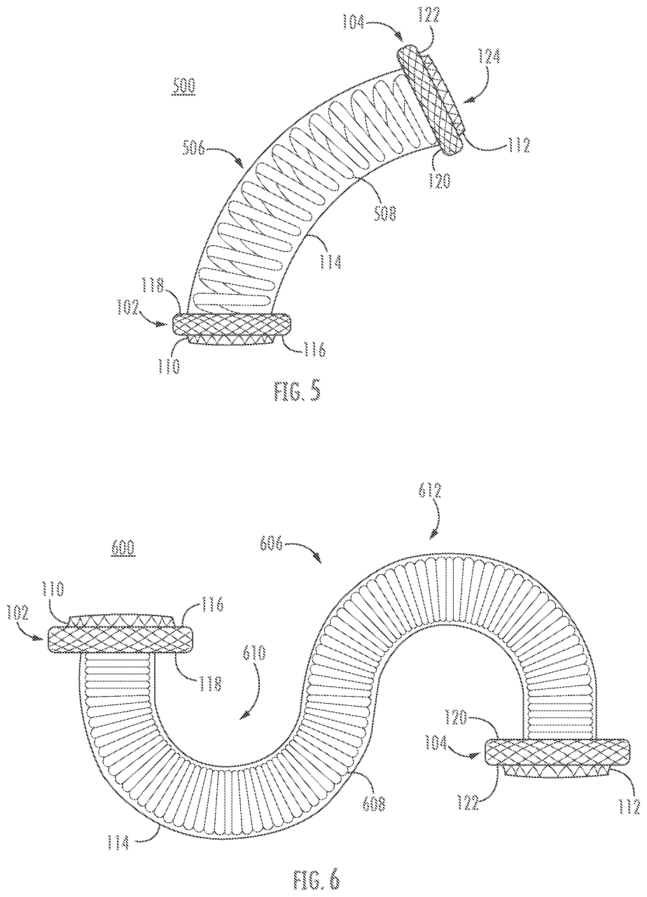

An elongated body, hollow body, central region, or saddle region, such as saddle region 106, may extend between the first and second retention members 102, 104 of the stent 100. Saddle region 106 may comprise a tubular structure, which in some embodiments may be cylindrical and linear in form and/or may comprise one or more curves (see FIGS. 5-6). A lumen 124 may extend along the longitudinal axis A-A of the stent 100 along an entire length of the stent (e.g., through first retention member 102, saddle region 106, and second retention member 104). In many embodiments, lumen 124 may be configured to enable flow of fluid through stent 100 so that fluid may drain or move between the tissues coupled to the stent.

In various embodiments, the first and/or second retention members 102, 104 may comprise a lip or projection 110, on at least one end thereof. The lip or projection 110 (collectively referred to herein, for brevity, as “lip”) may serve to guide flow therethrough and/or may provide a surface for a user to grip during placement and/or removal of the stent 100. In the illustrated embodiment, lip 110 of first retention member 102 and/or lip 112 of second retention member 104 may extend longitudinally along axis A-A away from the respective flange of the retention member. Lip 110, 112 may comprise a diameter that is the same, less than, or greater than a diameter of the saddle region 106, lumen 124, or corresponding first or second retention member 102, 104.

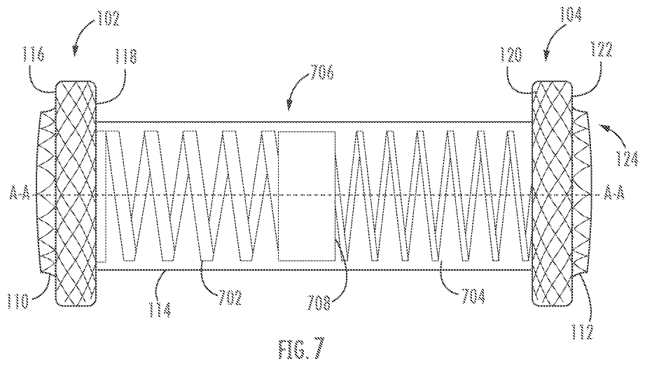

The saddle region 106 may include at least one spring member, such as coil 108. Coil 108 may extend along part of saddle region 106 or along an entire length of the saddle region. Coil 108 may be formed of flat wire, round wire, or a combination thereof. Coil 108 may comprise a single wire or multiple wires, and may be made of metal, polymer, or other appropriately elastic material. In many embodiments, coil 108 comprises a shape memory material, a non-limiting example of which is Nitinol. In various embodiments, coil 108 may be formed separately from first and second retention members 102, 104 of stent 100. For example, coil 108 may be formed and subsequently attached to one or more of retention members 102, 104 via glue, welding, or other appropriate attachment method. Alternatively, coil 108 may be continuously formed with one or more of the first and second retention members 102, 104 (e.g., formed by at least one wire of first and/or second retention members) such that the coil is integral to the first and/or second retention members. In some embodiments the coil 108 is formed of the same material used to form the first and second retention members 102, 104, while in other embodiments the coil is formed of a different material from that of the first and/or second retention members. While coil 108 is illustrated as substantially forming saddle region 106, saddle region 106 may additionally include one or more portions of a braided and/or woven saddle portion in addition to coil 108, such as described with respect to hoop 708 of FIG. 7. In some embodiments, coil 108 may be embedded in a stent weave pattern (not shown).

Various stents described herein may comprise at least one coil 108 having a relaxed configuration (i.e., where little or no external force is applied) and a deflected configuration (i.e., where external compression or elongation forces are applied). In many embodiments, the relaxed configuration may be preset by the application of heat. Thereafter, compression or expansion of coil 108 along lateral axis A-A can impart a desired bias between the first and second retention members 102, 104. In other words, the coil 108 may generate a restoring force that tends to return the coil 108 and the first and second retention members 102, 104 to a predetermined position. FIG. 1A shows stent 100 in a first configuration, in which the first and second retention members 102, 104 have been separated to expand the coil 108 along the longitudinal axis A-A. As a result, in the first configuration the coil 108 imparts a bias to the first and second retention members 102, 104 that tends to move the first and second retention members toward each other in the direction of arrows A to assume the second configuration shown in FIG. 1B or 1C. For example, stent 100 may move from the first configuration of FIG. 1A, through the intermediate configuration shown in FIG. 1B in the direction of arrows A, toward the compressed configuration shown in FIG. 1C. As will be appreciated, this may allow the stent 100 to adjust the distance between the first and second retention members 102, 104) to accommodate various anatomical geometries. For example, the stent 100 may automatically adjust its length to bridge tissue layers of various thicknesses (not shown).

As mentioned, the stent 100 may be configured to bias the first and second retention members 102, 104 either toward each other, or away from each other. For example, the first configuration of FIG. 1A may be a relaxed configuration while the second configuration of FIG. 1C may be a compressed configuration. Alternatively, the second configuration of FIG. 1C may be a relaxed configuration and the first configuration of FIG. 1A may be an extended configuration. In still other embodiments, the intermediate configuration of FIG. 1B may be a relaxed configuration, while the configurations of FIGS. 1A and 1C may be extended (FIG. 1A) or compressed (FIG. 1C) configurations in which the first and second retention members 102, 104 are biased so that they tend to return to the relaxed configuration of FIG. 1B.

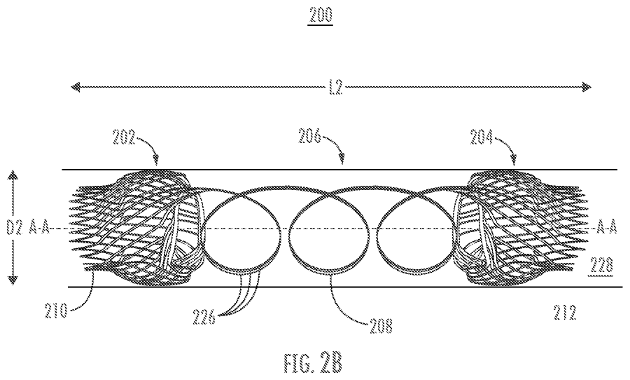

In some embodiments, first and/or second retention members 102, 104 may be configured to appose tissue, such as first and second tissue layers 308, 310 as described with respect to FIG. 3, so that the tissue portions are pressed together or held apart, depending on the requirements of the particular procedure. Thus, the first and second retention members 102, 104 may be configured to translate the force generated by the coil 108 (either from compression or expansion) to tissue apposed by the first retention member, tissue apposed by the second retention member, or both. In some embodiments, the coil 108 may be configured to effect a desired spacing between tissue layers via the first and second retention members 102, 104. In some embodiments, first and second retention members 102, 104 may be configured to be deployable within a single body lumen (e.g., in order to effect a diameter of a body lumen along the length of stent 100, not shown).

For example, first and/or second outer surfaces 116, 122 may be configured to appose respective tissue surfaces such that the first and second retention members 102, 104 are biased laterally outward (e.g., along arrows B in FIG. 1C). Accordingly, stent 100 may be positioned between two tissue layers to apply a force to the tissue layers, either to press them together (e.g., to improve a seal between the respective first and/or second outer surface 116, 122 and apposed tissue) or to hold them apart (e.g., to hold a diseased tissue layer away from a healthy tissue layer, and/or to provide a space between the two tissues for accessibility for another procedure or application).

In some non-limiting example embodiments, first and/or second inner surfaces 118, 120 of the first and second retention members 102, 104 may appose respective tissue surfaces to press the tissue surfaces together. In other non-limiting example embodiments, first and/or second outer surfaces 116, 122 of the first and second retention members 102, 104 may appose respective tissue surfaces to hold the tissue surfaces apart. In either instance, the tissue layers may be effectively bridged by stent 100, and the stent may create a fluid flow path therebetween.

In many embodiments, a covering, coating, or other layer (collectively referred to herein, for brevity, as cover 114) may be applied to or may be otherwise coupled to the stent 100 to encapsulate at least a portion of the stent. As will be appreciated, providing a covering for at least a portion of the stent 100 may prevent tissue from snagging, catching, or otherwise engaging portions of the stent in a manner that may interfere with a desired operation. Cover 114 may comprise, for example, silicone or other polymer material, or a combination thereof. For example, the cover 114 may comprise polyurethane, polytetrafluoroethylene, polyvinylidene fluoride, an aromatic polycarbonate-based thermoplastic urethane, and/or another material, or the like. Cover 114 may be applied to the stent 100, such as by dip coating, painting, spraying, or other known deposition method, or a combination thereof. Alternatively, cover 114 may be separately formed from the stent 100, such as in a sheet or sleeve, and may be subsequently positioned around and attached to the stent, such as by glue, one or more ties, shrink fit, or other appropriate method of attachment. In some embodiments, cover 114 may be configured to stretch or otherwise deform or deflect in accordance with a corresponding deformation or deflection of stent 100. In some non-limiting examples, cover 114 may be configured to be less elastic than coil 108, such that the cover 114 may contribute to a biasing of the stent 100 to return to a relaxed configuration when the stent is deformed.

It will be appreciated that although the description of stent 100 in FIGS. 1A-C has proceeded thus far in relation to a coil 108 for biasing the first and second retention members 102, 104 together or apart, that other biasing members could be used in lieu of a coil, for example, an elastic band array, or the like.

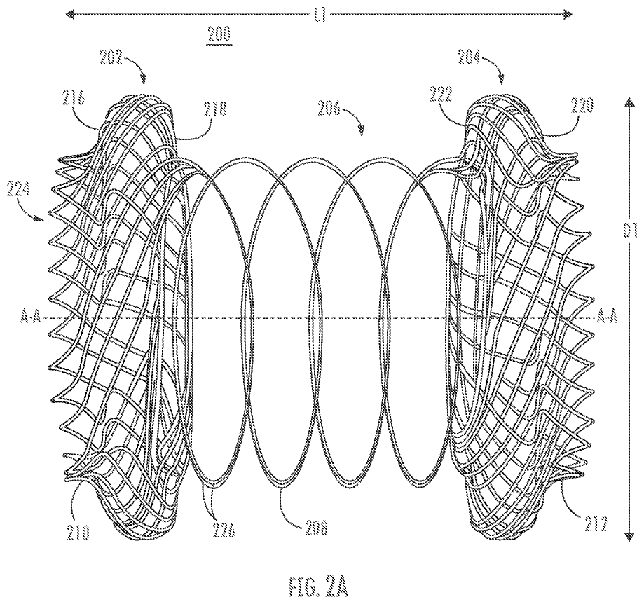

Referring to FIGS. 2A-B, an exemplary coil arrangement will be described in greater detail. Stent 200 may comprise a first retention member 202, second retention member 204, and a saddle region 206 comprising a coil 208 that is made of one or more wires 226 used to form the first and second retention members. In this manner, coil 208 is formed integrally to the first and second retention members 202, 204. FIG. 2A shows stent 200 in a diametrically expanded configuration, while FIG. 2B shows stent 200 in a compressed configuration.

In an expanded configuration, first and/or second retention members 202, 204 of stent 200 may be configured to appose tissue layers similarly to first and second retention members 102, 104 described with respect to FIGS. 1A-C. In contrast to the embodiment of FIGS. 1A-C, first and second outer surfaces 216, 220 and first and second inner surfaces 218, 222 of the first and second retention members 202, 204 comprise curved surfaces. In some examples, providing curved inner and outer surfaces on the first and second retention members 202, 204 may enable the members to engage respective tissue layers atraumatically or with reduced risk of abrasion to the tissue layers as compared to flat or angled surfaces. The first and second retention members 202, 204 may include respective lips 210, 212 that extend along axis A-A in a direction longitudinally away from the respective retention members 202, 204, similar to lips 110, 112 described with respect to FIGS. 1A-C.

Retention members 202, 204 may be formed of one or more wires 226, which may be formed into coil 208 along saddle region 206. For example, a single wire 226 may be wrapped, braided, or woven into the first and second retention members 202, 204 such that a portion or portions of the wire 226 extend therebetween. The wire 226 may then be gathered and/or twisted to form coil 208. In another example, multiple wires 226 may be braided, woven, or formed into a mesh structure to form first and/or second retention members 202, 204. Portions of the wires 226 extending between the first and second retention members 202, 204 may then be gathered and/or twisted to form coil 208. Some or all of the wires 226 used to form first and/or second retention members 202, 204 may also form coil 208. In another example (not shown), stent 200 may be made of a single wire 226, which may be woven to form first retention member 202, extended into a coil 208 along axis A-A, and then woven at a second end to form second retention member 204.

Coil 208 may function similarly to coil 108 as described with respect to FIGS. 1A-C. Thus, coil 208 may be configured so that the first and second retention members 202, 204 are biased apart (such as in FIG. 1A), biased together (such as in FIG. 1C), or biased to an intermediate position (such as in FIG. 1B). It will be understood that the stiffness of coil 208 (e.g., its retentive strength, restoring force, or biasing force) may be increased, for example, linearly, by increasing the number of wires and/or thickness of wires used to form the coil. Accordingly, stent 200 may be configured to have a predetermined biasing force for a particular application or for use with particular types of tissues. In some embodiments the stent 200 may have a spring force in the range described in relation to the stent 100 described in relation to FIGS. 1A-C.

Saddle region 206 may extend between first and second retention members 202, 204 similar to saddle region 106 described with respect to FIGS. 1A-C. Lumen 224 may extend along a full length of stent 200 (“L1”), to provide a flow path therethrough so that fluids may move through the stent. While, for the sake of simplicity, stent 200 is illustrated without a cover in FIGS. 2A-B, it will be understood that the stent may comprise a coating or covering as described with respect to stent 100 of FIGS. 1A-C.

In many embodiments, stents described herein may be delivered to a patient tissue site in a radially compressed or constrained state (referred to as a delivery configuration) so as to be contained within the lumen 228 of a catheter or other delivery device. When the stents are positioned within the patient, they may be released from the delivery device and may assume an expanded, unconstrained, or deployed, configuration. Stents may be self-expanding stents or may be expanded, for example, by a balloon. An expanded configuration of stent 200 may be preset (e.g., via heat setting) so that the stent 200 has a predetermined length L1 and a predetermined diameter “D1” in the expanded or deployed configuration. FIG. 2B shows stent 200 in an exemplary constrained configuration, with a diameter “D2”, that is smaller than D1, for example, so that the stent 200 can be disposed within the delivery device lumen 228. In the delivery configuration, stent 200 may lengthen along longitudinal axis A-A to assume a length “L2” that is greater than L1. As will be appreciated, any or all of the components of stent 200 may lengthen longitudinally and/or reduce radially when the stent is in the constrained configuration. It is noted that FIG. 2B shows the individual elements of stent 200 as having distinct radial dimensions (e.g., lip 210 as distinctly smaller in diameter compared to retention member 202). It will be understood, however, that stent 200 may have a constrained configuration in which the individual elements are substantially constant in diameter along a length of the stent. In some embodiments, the coil 208 may be expanded or stretched in when in the constrained configuration so as to have a smaller diameter and/or to approach a linear shape. Stent 200 may be configured to move automatically from a constrained configuration to an expanded configuration upon release from constraint, such as where the stent is formed of a shape memory material. It will be appreciated that the stent 100 described in relation to FIGS. 1A-C may include delivery and deployed configurations similar to those described in relation to stent 200.

FIG. 3 shows an exemplary deployment sequence for stent 100 according to one or more embodiments described herein. While, for the sake of simplicity, methods are described as relating to stent 100, it will be understood that methods may apply to any stent described herein.

As mentioned, a delivery shaft, such as a catheter 316 may be used to deliver stent 100 in the constrained delivery configuration to a desired location in or adjacent to patient tissue. Stent 100 may be disposed about an inner member 302 of the catheter 316 when the stent is deployed from the catheter (i.e., beyond the outer member 304, which may hold the stent 100 in a radially compressed delivery configuration). As can be seen, the inner member 302 is disposed through the lumen 124 of the stent 100 such that tip 306 of the catheter is disposed beyond the stent. In some embodiments the tip 306 may be a tissue cutting element such as an electrocautery tip.

Inner member 302, outer member 304, and the stent 100 may be advanced together in the direction of arrow C through first and second openings 318, 320 formed in first and second tissue layers 310, 308. Thus arranged, the first retention member 102 is disposed in body lumen 312 associated with first tissue layer 308, while the second retention member 104 is disposed in body lumen 314 associated with second tissue layer 310. When outer member 304 is retracted in the direction of arrow D the first retention member 102 expands within body lumen 312, and the second retention member 104 expands within body lumen 314, with coil 108 disposed therebetween. To facilitate appropriate placement of stent 100, the inner member 302 and/or outer member 304 may include one or more echogenic, radiopaque, and/or visual markers.

As shown, the stent 100 bridges the first and second tissue layers 308, 310. Coil 108 is configured to bias the first and second retention members 102, 104 toward each other. Accordingly, coil 108 applies a force, via first and second inner surfaces 118, 120, to respective first and second tissue layers 308, 310 to draw the tissue layers together. The deployment system including inner member 302, outer member 304, and tip 306 may be removed through the lumen 124 leaving the stent 100 in place between the first and second tissue layers 308, 310.

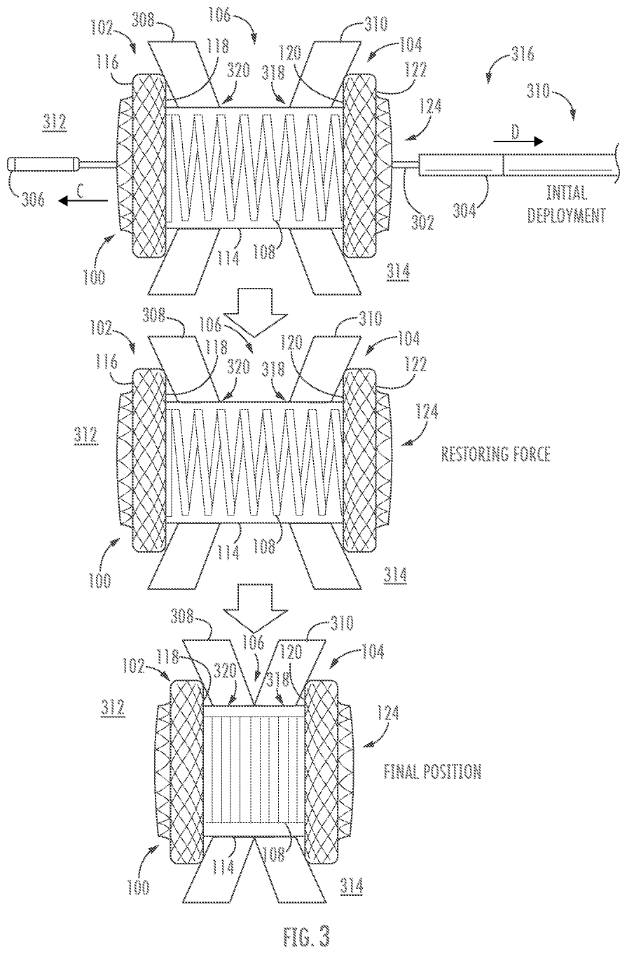

FIG. 4 shows a stent 400 disposed in tissue according to one or more embodiments. Stent 400 comprises one or more features similar to those described above in relation to stents 100 and 200, such as saddle region 106, coil 108, cover 114, and lumen 124. In the embodiment of FIG. 4, however, retention members 402 and 404 comprise multi-flanged structures, which may more securely appose tissue layers 308, 310 by interacting with opposing sides of each tissue layer. For example, retention member 402 may comprise a first flange 422 with surfaces 406, 408 and a second flange 424 with surfaces 410 and 412. Retention member 404 may comprise a third flange 426 with surfaces 414, 416 and fourth flange 428 with surfaces 418, 420. Any of surfaces 406, 406, 410, 412, 414, 416, 418, and 420 may be configured to appose tissue, but in many embodiments, multi-flanged retention members may be configured to hold tissue layers between flanges of each retention member. Accordingly, surfaces 408, 410 may be deployed to appose first tissue layer 308 and surfaces 416, 418 may be deployed to appose second tissue layer 310. Stent 400 may be deployed using methods similar to those described with respect to FIG. 3, except that first flange 422, second flange 424, third flange 426, and fourth flange 428 may be sequentially deployed to appose respective tissue layers. As illustrated in FIG. 4, the stent 400 bridges the first and second tissue layers 308, 310. Coil 108 is configured to bias the first and second retention members 102, 104 away from each other. Accordingly, coil 108 applies a force, via surfaces 410, 416, to respective first and second tissue layers 308, 310 to push the tissue layers apart (e.g., along arrows E). The deployment system (not shown) may be removed leaving the stent 400 in place between the first and second tissue layers 308, 310. It will be understood that one or both of retention members 402, 404 may alternatively comprise fewer or more than two flanges each (not shown).

FIGS. 5-6 show alternative stent configurations according to various embodiments described herein. For the sake of simplicity, stents 500, 600, respectively of FIGS. 5-6, may have the same or similar features and functionalities of stent 100 described with respect to FIGS. 1A-C, and may alternatively or additionally comprise dual-flange retention members 402, 404 as described with respect to FIG. 4, with the exception that saddle regions 506, 606 of stents 500, 600 may have non-linear shapes. As can be seen, saddle region 506 of FIG. 5 is curved along its length, while saddle region 606 of FIG. 6 includes a pair of curved portions 610, 612. Curved stents 500, 600 may be useful for curving around vessels and organ surfaces or regions that may be difficult to reach. It will be appreciated that these are but two non-limiting examples of curved stents, and that stents according to the disclosure may comprise any number of curves, turns, bends, or the like, including the same or differing radii of curvature, corresponding inflection point(s), or the like. It will be understood that radii of curvature of curved portions 506, 610, and 612 may be optimized in coordination with a spring constant, length, or other aspect of stent 500, 600 to accommodate corresponding stresses that the stent 500, 600 may encounter post-placement. Curves may be preset in an expanded configuration (e.g., via heat setting). The coils 508, 608 of stents 500, 600 are illustrated as comprising round wires, but it will be understood that flat wires, gathered wires, or any other coil construction as disclosed herein may comprise one or more curves.

FIG. 7 shows an additional embodiment according to the present disclosure, wherein stent 700 comprises a saddle region 706 containing multiple coils 702, 704. Stent 700 may have the same or similar features and functionalities of stent 100 described in relation to FIGS. 1A-C, with the exception that saddle region 706 may comprise two or more coils 702, 704, which in many embodiments may comprise different thicknesses, spring constants, resistances to deformation, restoring forces, diameters, or other properties. For example, first coil 702 is illustrated as being formed from a thicker wire than second coil 704, and thus first coil 702 may have a larger spring force than coil 704. Coils of varying restoring forces may be useful, for example, for use in body passages that have different applied pressures or geometries. For example, the portion of stent 700 corresponding with second coil 704 may be more easily deflectable about longitudinal axis A-A than the portion corresponding with first coil 702, and thereby the stent 700 may be more suitable for use within a portion of a tortuous body passage. It will be appreciated that differing spring constants between the first and second coils 702, 704 may also or alternatively be implemented by using different materials for each, in lieu of providing coils with different wire thicknesses. In addition, although two coils are illustrated, it is contemplated to greater numbers of coils may also be used. Further, a single coil having a continuously varying, and/or stepped, dimension could also be used to provide a stent having a variable stiffness along its length.

The first and second coils 702, 704 of stent 700 may be coupled to each other directly by welding, adhesive, or the like, or they may be coupled via one or more connecting members, such as rings, hoops, or the like, while maintaining an open passage of the lumen of the stent. The first and second coils 702, 704 comprise the same or different lengths and may be joined at a center point or elsewhere along a saddle region. For example, in the illustrated embodiments the first and second coils 702, 704 are each coupled to hoop 708 at an approximate midpoint of the stent 700. It will be understood that coils may be coupled end-to end or in series, as shown in FIG. 7, or in tandem or parallel about a common or separate lumen (not shown). While hoop 708 is illustrated as having a continuous (e.g., solid) circumference, it will be understood that hoop 708 may alternatively comprise a braided or woven stent saddle portion, which may be formed continuously or separately from coil 702 and/or coil 704. Various coils of a stent may also be made of the same or different materials. Embodiments are not limited herein.

It will be understood that various embodiments may comprise alternative retention member designs. While not illustrated for the sake of brevity, a stent may comprise asymmetric flange designs, for example, with a first retention member 102 and a second retention member 404. In another example (not shown), a stent may comprise a first retention member 102 but not a second retention member 104. It will be understood that any combination of features described herein is within the scope of the present disclosure.

Embodiments described herein may comprise various dimensions and/or properties suitable for a variety of purposes and/or uses with respect to various tissues. For example, a stent may comprise a length of a saddle region of 5-40 mm in a deployed configuration.

In some embodiments, the saddle region of a stent in the constrained configuration, such as saddle region 106, may comprise a diameter in the range inclusive of 2-6 mm, or 3-5 mm. For example, a stent may have a diameter D2 in a constrained configuration of 10Fr or 3.5 mm. In some embodiments, the saddle region in the constrained configuration may have a length within the range inclusive of 40-150 mm. For example, a stent in a constrained configuration may have a length L2 within the range inclusive of 40-100 mm. In some embodiments, a stent in the constrained configuration may have a length L2 within the range inclusive of 40-80 mm.

The saddle region may comprise a length and a diameter. In embodiments, such as stents having double-walled flanges as first and second retention members, the length of the saddle region may be measured as (i) the length along the elongate body between the beginning of the inward surfaces of each of the flanges, or (ii) the length along the body that is the shortest distance between flanges at any points along the inward surfaces when the stent is expanded, but not deployed in tissue, or (iii) the length along the body that is the shortest distance between flanges at any points along the inward surfaces when the stent is expanded and deployed in tissue. In many embodiments, a saddle region, including under the conditions (i)-(iii) above, may have a length within the range inclusive of 5-150 mm in the expanded configuration, although in some instances the saddle region (such as saddle region 106) may have a length in the range inclusive of 5-40 mm or 5-20 mm in the expanded configuration. Exemplary lengths of the saddle region of devices for gastrointestinal stenting or drainage in the expanded configuration may include lengths within a range inclusive of 10-30 mm, 10-20 mm, 10-15 mm, or 5-10 mm.

In many embodiments, a diameter of the saddle region in the expanded configuration may be greater than a diameter of the elongate body in the constrained configuration. For example, a diameter of the saddle region (such as saddle region 106) in the expanded configuration may be within the range inclusive of 3-40 mm. In some embodiments, the diameter of a saddle region in the expanded configuration may be within inclusive ranges of 5-25 mm, 5-20 mm, or 10-20 mm. In various embodiments, the diameter of the saddle region in the expanded configuration may be 3-5 times greater than a corresponding diameter of the stent in the constrained configuration, although in several embodiments, the diameter of the saddle region in the expanded configuration may be 3-10 times greater than the corresponding diameter of the stent in the constrained configuration.

A lumen of a saddle region, in an expanded configuration, such as lumen 124 as described with respect to FIGS. 1A-C, may include, for example, an inner diameter of 3-25 mm.

In embodiments, a diameter of the first retention member and/or a second retention member (such as first and second retention members 102, 104 as described with respect to FIGS. 1A-C or retention members 402, 404 as described with respect to FIG. 4) may be within the range inclusive of 5-40 mm. Other exemplary retention member diameters may be within a range inclusive of 15-40 mm or 15-35 mm. In many embodiments, a diameter of a retention member may be set to have a particular offset from a diameter of the saddle region in the expanded configuration. For example, stents may be configured to include a 3-20 mm difference between a diameter of the saddle region and a larger diameter of the first retention member and/or a second retention member in the expanded configuration. Some embodiments may include a 6-10 mm difference between an outer diameter of the saddle region and a larger outer diameter of a retention member. For example, for an outer diameter of a saddle region of 10 mm, an outer diameter of a proximal flange and/or a distal flange may be between 16-20 mm. It will be understood that some embodiments may include greater differences between an outer diameter of a saddle region and an outer diameter of a proximal flange and/or a distal flange. In some embodiments, such as in esophageal stents, a difference between an outer diameter of a saddle region and an outer diameter of a proximal flange and/or a distal flange may be 4-6 mm. Other embodiments may comprise larger and/or smaller differences between an outer diameter of a saddle region and an outer diameter of a proximal flange and/or a distal flange. In other examples, a retention member may be configured to have a diameter that is 1-5 times greater than the diameter of the saddle region in the expanded configuration. For example, for a device with the saddle region having a diameter of 10 mm in an expanded configuration, the first retention member and/or a second retention member may have a diameter within inclusive ranges of 13-30 mm, 15-25 mm, or 16-20 mm. In another example, a stent with the saddle region in the expanded configuration having a diameter of 20 mm may have one or more retention members with a diameter within the inclusive range of 23-40 mm. It will be understood that some embodiments may include greater or lesser offsets between the diameter of the saddle region and the larger diameter of the first retention member and/or the second retention member.

A first retention member, such as first retention member 102, may have a same or different axial width as a second retention member, such as second retention member 104, wherein the axial width of a retention member may be measured as a distance along the longitudinal axis between an inward surface and outward surface of a respective retention member. In some embodiments, the width of the first retention member and/or the second retention member in the expanded configuration may be within the inclusive range of 0.5-10.0 mm. In some embodiments, a width of a first and/or second retention member may be within the inclusive range of 0.5-3.0 mm. Other embodiments may include a smaller and/or greater width of the first retention member and/or the second retention member, such as within the inclusive rages of 0.5-6 mm, 2-6 mm, or 3-7 mm. In some embodiments, a retention member may have a constant width. In other embodiments, a width of a retention member may vary along a vertical plane.

In some embodiments, a proximal protruding end and/or distal protruding end, such as lips 110, 112 as described with respect to FIGS. 1A-C, may have a length within the range inclusive of 0-3 mm in the expanded configuration, such as within the inclusive ranges of 0.5-2.5 mm or 1.0-1.5 mm. The proximal protruding end and/or distal protruding end may define a lumen, such as a flange lumen contiguous with the saddle region, with a diameter greater than or equal to the diameter of the lumen defined by the saddle region, or saddle lumen. In various embodiments, a lip, or projection, may comprise a flange lumen diameter in the expanded configuration that is at least 0.5-2.0 mm wider than the diameter of a corresponding saddle region in an expanded configuration, for example, within the diameter range inclusive of 3.5-42 mm for a saddle diameter within the range inclusive of 3-40 mm. As further exemplary inclusive ranges, for a corresponding saddle region diameter of 10 mm in the expanded configuration, a lip may have a diameter of 11-14 mm. For a corresponding saddle region diameter of 15 mm in the expanded configuration, a lip may have a diameter of 16-19 mm. In another example, a saddle region diameter of 20 mm in the expanded configuration may correspond with a lip diameter of 21-24 mm. A lip may be formed contiguously with the retention members and/or with the saddle lumen. The lip may be parallel to the longitudinal axis of the saddle region. Alternatively, or additionally, the lip may be non-parallel to the longitudinal axis. For example, the lip may extend radially outward or inward along the longitudinal axis. A radially outward extension of the lip may encourage flow through a lumen thereof, for example, by acting as a funnel into the lumen. A radially inward extension of the lip may discourage flow through a lumen thereof, for example, by defining a smaller entrance thereof.

Various embodiments may include total stent lengths ranging from 5-60 mm in the expanded configuration. For example, exemplary deployed stents may have lengths of 10-50 mm or 10-40 mm.

In various embodiments, a coil such as coil 108, 208, 508, 608, 702, and/or 704 may be configured with a spring constant suitable for treatment of and/or placement with respect to one or more tissues or body lumens. In many embodiments, a coil may have a spring constant in the range of 50-200 N/m. It will be understood that a coil may have a spring constant relating to its geometry, for example, relating to a thickness of the coil.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A stent, comprising:
an elongate body having a constrained configuration,
the elongate body having an expanded configuration with a first portion expanded into a first retention member, a second portion expanded into a second retention member, and a saddle region defining a lumen extending between the first and second retention members;
wherein the saddle region is formed of a coil configured to apply a restoring force to tissue surfaces respectively apposed to the first and second retention members.

2. The stent of claim 1, wherein the first retention member comprises a first flange and a second flange configured to appose first and second surfaces of the first tissue, wherein the second retention member comprises a third flange and a fourth flange configured to appose first and second surfaces of the second tissue, or both.

3. The stent of claim 1, wherein the first retention member comprises a mesh structure, the second retention member comprises a mesh structure, or both.

4. The stent of claim 1, wherein the coil extends along an entire length of the saddle region.

5. The stent of claim 1, wherein the coil is configured to apply tension between the first retention member and the second retention member.

6. The stent of claim 1, wherein the coil is configured to apply a laterally outward force between the first retention member and the second retention member.

7. The stent of claim 1, wherein the saddle region comprises two or more coils.

8. The stent of claim 7, wherein each of the two or more coils comprise different spring constants.

9. The stent of claim 1, wherein saddle region comprises at least one curve.

10. The stent of claim 1, wherein the first retention member, the second retention member, and the coil are continuously formed of wire.

11. The stent of claim 1, wherein the first retention member comprises a first surface substantially perpendicular to a longitudinal axis of the saddle region, the first surface configured to appose a first tissue, wherein the second retention member comprises a second surface substantially perpendicular to a longitudinal axis of the saddle region, the second surface configured to appose a second tissue, or both.

12. A stent, comprising:
a hollow body comprising a constrained configuration and an unconstrained configuration, the hollow body in the unconstrained configuration comprising a first retention member, a second retention member, and a central region therebetween,
wherein the central region comprises a coil configured to, when deflected, generate a force to return towards a predetermined position, and
wherein the first retention member and the second retention member are configured to translate the force to tissue engaged by the first retention member, tissue engaged by the second retention member, or both.

13. The stent of claim 12, wherein the coil is configured to apply tension between the first retention member and the second retention member.

14. The stent of claim 12, wherein the coil is configured to apply a laterally outward force between the first retention member and the second retention member.

15. The stent of claim 12, wherein the first retention member, the second retention member, and the coil are continuously formed of wire.

16. A stent, comprising:
a first retention member configured to appose a first tissue layer;
a second retention member configured to appose a second tissue layer; and
a saddle region extending therebetween, wherein the saddle region comprises a coil configured to affect a spacing between the first tissue layer and the second tissue layer.

17. The stent of claim 16, wherein the coil is configured to apply tension between the first retention member and the second retention member.

18. The stent of claim 16, wherein the coil is configured to apply a laterally outward force between the first retention member and the second retention member.

19. The stent of claim 16, wherein the first retention member, the second retention member, and the coil are continuously formed of wire.

20. The stent of claim 16, wherein the coil extends along an entire length of the saddle region.
